(19) United States
(12) Patent Application Publication
(10) Pub. No.: US 2004/0255959 A1
(43) Pub. Date: Dec. 23, 2004

(54) Method and compounds to decrease the incidence of atrial fibrillation (AF) in patients with left ventricular dysfunction

(76) Inventors: Anique Ducharme, Montreal (CA); Jean-Claude Tardif, Laval (CA); Martial G. Bourassa, Iles Laval (CA)

Correspondence Address: Ms. Diane Dunn McKay, MATHEWS, COLLINS, SHEPHERD & McKAY, P.A., Suite 306, 100 Thanet Circle, Princeton, NJ, 08540-3674, US

(21) Appl. No.: 10/826,240

(22) Filed: Apr. 15, 2004

Related U.S. Application Data
(60) Provisional application No. 60/462,734, filed on Apr. 15, 2003.

(51) Int. Cl. A61N 1/18; A61N 1/20

(52) U.S. Cl. 128/898; 604/500

(57) ABSTRACT

Atrial fibrillation (AF) is frequently encountered in patients with heart failure (HF) and is also a predictor of morbidity and mortality in this population. Recent experimental studies have shown electrical and structural atrial remodeling with increased fibrosis in HF animals, and have suggested a preventive effect of angiotensin converting enzyme inhibitors (ACEi) on the development of AF. To verify the hypothesis that ACEi prevent the development of AF in patients with HF, a retrospective analysis of the patients from the Montreal Heart Institute (MHI) included in the Studies Of Left Ventricular Dysfunction (SOLVD) was conducted. The results of this retroactive analysis indicate that treatment with the ACE inhibitor, such as enalapril, can markedly reduce the risk of developing atrial fibrillation in patients with left ventricular dysfunction.

### FIELD OF THE INVENTION

The present invention relates to the prevention of atrial fibrillation (AF) in a subject with left ventricular dysfunction. Specifically, the present invention concerns the use of an angiotensin-converting enzyme (ACE) inhibitor, such as enalapril, to lessen the chances that a subject with left ventricular dysfunction, whether symptomatic or not, will develop AF.

### BACKGROUND OF THE INVENTION

Atrial fibrillation (also called AF or A Fib) is the most common abnormal heart rhythm. It is a very fast, uncontrolled heart rhythm caused when the upper chambers of the heart (the atria) quiver instead of beating. During AF, the upper chambers of the heart beat between 350 and 600 times per minute. Normal heart rhythm is between 60 and 100 beats per minute. Since the pumping function of the upper chambers isn't working properly, the blood is not completely emptied from the heart's chambers, causing it to pool and sometimes clot. In approximately 5% of patients with AF, clotted blood dislodges from the atria and results in a stroke. The American Heart Association estimates that in the United States, AF is responsible for over 70 000 strokes every year.

AF has three stages. Paroxysmal AF is characterized by brief episodes of the arrhythmia which resolve themselves. In persistent AF, the episodes require some form of intervention to return the heart rhythm back to normal. With permanent AF, intervention (if successful at all) only restores normal heart rhythm for a brief time.¹

Atrial fibrillation is a common finding in patients with heart failure (HF), its prevalence increasing with the severity of the disease and reaching 40% in advanced stages.^{2,3} AF may also cause patients with congestive HF to decompensate as evidenced by a decline in cardiac index and peak oxygen consumption and worsening of functional class when AF occurs in these patients.⁴ In this population, the presence of AF is an independent predictor of morbidity and mortality,⁵⁻⁷ increasing the risk of death and cardiovascular hospitalization by 76%.⁶ AF occurring in the course of experimental HF-induced by rapid ventricular pacing is accompanied by atrial electrical and structural remodeling, including atrial dilation, contractile dysfunction and fibrosis.^{8,9} Recent experimental studies have demonstrated a role for angiotensin-converting enzyme inhibitors (ACEi) in the prevention of this atrial structural remodeling.^{10,11}

Notwithstanding the above, the impact of chronic ACEi therapy on the incidence of AF in patients with established left ventricular dysfunction has remained unanswered.

### SUMMARY OF THE INVENTION

In order to determine the impact of chronic ACEi therapy on the incidence of AF in patients with established left ventricular dysfunction, a retrospective analysis of the Montreal Heart Institute patients randomized in SOLVD (Studies Of Left Ventricular Dysfunction) was conducted. Specifically, the impact of the ACEi enalapril on the incidence of AF in this population was examined. The results of this analysis show that enalapril markedly reduces the risk of developing AF in patients with left ventricular dysfunction, whether symptomatic or not.

In accordance with the present invention therefore, a method is described for reducing the incidence of atrial fibrillation (AF) in subjects with left ventricular dysfunction. This method comprises the administration of an ACEi, such as enalapril, to a subject suffering from left ventricular systolic dysfunction (whether symptomatic or not) or hypertension, or an individual afflicted with another heart ailment who is predisposed to AF.

In an embodiment of the present invention, the ACE inhibitor is chosen from the following group: enalapril (Vasotec®), captopril (Capoten®)), lisinopril (Prinivil®, Zestril®), quinapril (Accupril®), ramipril (Altace®), trandolapril (Mavick®), perindopril (Coversyl®)), and fosinopril (Monopril®). The ACE inhibitor enalapril is administered in a dosage of about 5-20 mg/day, as determined by the attending physician. Similarly, the ACE inhibitor captopril is administered in a dosage of about 150 mg/day; the ACE inhibitor lisinopril is administered in a dosage of about 20 mg/day; the ACE inhibitor quinapril is administered in a dosage of about 40 mg/day; the ACE inhibitor ramipril is administered in a dosage of about 10 mg/day; the ACE inhibitor trandolapril is administered in a dosage of about 4 mg/day; the ACE inhibitor perindopril is administered in a dosage of about 8 mg/day; and the ACE inhibitor fosinopril is administered in a dosage of about 20 mg/day.

Like ACE inhibitors, angiotensin 11 receptor antagonists have an effect on the renin-angiotensin system. ACE inhibitors exert their effects earlier in the renin-angiotensin pathway than do angiotensin 11 receptor antagonists. Given the similarities in modes of action and overall effects caused by individual members of these two classes of compounds, it is believed that an angiotensin II receptor antagonist might be effectively used as an alternative (or substitute) for an ACE inhibitor in the prevention of AF in a subject with chronic heart failure. Suitable angiotensin II receptors include the following: losartan (Cozaar®)), candesartan (Atacand®), irbesartan (Avapro®)), telmisartan (Micardis®), valsartan (Diovan®) and eprosartan (Teveten®).

Subjects who are most likely to benefit from the present invention include individuals suffering from left ventricular systolic dysfunction (whether symptomatic or not) or hypertension, as well as individuals with other heart ailments who are predisposed to AF.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

Figure Legends

FIG. 1: Kaplan-Meier curves of the percentage of patients remaining free of a first occurrence of AF during 2.9 years of follow-up in 374 patients with depressed left ventricular function and sinus rhythm at baseline randomized to enalapril (solid line) or placebo (dotted line) (P<0.0001).

FIG. 2: Kaplan-Meier curves for the time to first occurrence of AF in the subgroup of 251 patients of the prevention arm randomized to enalapril (solid line) or placebo (dotted line) (P<0.0001), including patients with LVEF≦0.35 and no history of overt HF requiring treatment at entry in the trial.

FIG. 3: Kaplan-Meier curves for the time to first occurrence of AF in the subgroup of 123 patients of the treatment arm randomized to enalapril (solid line) or placebo (dotted line) (P=0.062), including patients with a history of overt heart failure requiring treatment for symptomatic relief.

### DESCRIPTION OF THE PREFERRED EMBODIMENTS

Methods

Study Population

The patients of the Montreal Heart Institute who were enrolled in the SOLVD trials constituted the study population. SOLVD was a multicenter, double-blinded, randomized, placebo-controlled trial which evaluated the effect of the ACEi enalapril on survival in patients with left ventricular (LV) dysfunction (ejection fraction≦35%).^{12,13} The design of the study has been reported previously.¹³ Briefly, from June 1986 to August 1991, 4228 patients with asymptomatic or mildly symptomatic (not requiring treatment with digitalis, diuretics or vasodilators for heart failure at study entry) LV dysfunction (LVEF≦0.35) were included in the prevention trial and 2569 patients with overt congestive HF in the treatment trial.¹⁴ Patients were randomized to enalapril (5-20 mg/day) or placebo. Exclusion criteria included: age>80 years, unstable angina, myocardial infarction in the previous month, severe pulmonary disease, renal insufficiency (creatinine level>177 mmol/l), current ACEi use or intolerance to ACEi. Follow-up visits were scheduled 2 and 6 weeks after randomization and every 4 months until the end of the study, for a mean follow-up of 3.4 and 3.1 years for the treatment and prevention trials, respectively.

Data Collection and Definitions

Baseline characteristics, past medical history and drug therapy at the time of enrolment were obtained from the SOLVD databases. Serial electrocardiograms (ECG) were not collected specifically for the SOLVD trials. However, the routine clinical follow-up of patients at our institution usually included a 12-lead ECG. Thus, the medical file of each patient was carefully reviewed and a single experienced cardiologist, blinded to treatment allocation, interpreted every ECG.

AF was defined as rapid oscillations or fibrillatory waves that vary in size, shape and timing, associated with an irregular, frequently rapid ventricular response. For the purposes of the study, paroxysmal AF was defined as episodes in which the patient reverted to sinus rhythm spontaneously, with medical therapy orwith a single cardioversion, whereas patients who remained in AF despite changes in medical therapy and/or cardioversion were defined as persistent AF. Episodes occurring during a 24-hour Holter monitoring were also considered. The end-points were the development and time to first occurrence of AF on either one 12-lead ECG and/or a 24-hour Holter monitoring recorded during any available follow-up visits (including research, outpatient clinic or emergency room visits). Participants with significant supraventricular arrhythmia on the baseline ECG (AF or flutter) were excluded. Patients with a history of arrhythmia (either supraventricular or ventricular) but who were in sinus rhythm on the ECG at the time of randomization were included.

Statistical Analysis

The baseline characteristics of the two groups were compared using student t-test for continuous variables and chi-square test for categorical variables. The incidence of AF between the two groups was compared with the chi-square test. Time to the first occurrence of AF during the follow-up was analyzed with Kaplan-Meier curves and compared with the log-rank test. To analyze the effect of enalapril on development of AF, a Cox regression analysis was used to take into account the effect of potential confounding baseline variables (age, sex, NYHA class, history of supraventricular or ventricular arrhythmia, ischemic etiology, diabetes and ejection fraction) and time-dependent variables (systolic blood pressure, diastolic blood pressure, pulse pressure, serum potassium and drug therapy). Cox proportional-hazard models were performed for each variable with treatment (enalapril) forced in all models. Variables with a p-value≦0.2 were included in a multivariate Cox proportional hazard model. For time-dependent variables, the last value before the occurrence of AF was taken or, if the patient did not develop AF, the value at the last visit was used.

Subgroup analysis was conducted with chi-square test. Preliminary assumptions were verified prior to all analysis. A p-value≦0.05 was considered statistically significant. All analyses were performed using SAS version 8.2 (SAS Institute Inc., Carey, N.C., USA).

Results

Among the 391 patients from the Montreal Heart Institute who were randomized in SOLVD, 17 (4.3%) had significant arrhythmia on the baseline ECG at randomization (16 AF and one flutter). The remaining 374 patients constituted the study population: 251 in the prevention arm and 123 in the treatment arm. Of these, 186 were randomized to enalapril and 188 to placebo. The mean follow-up of the patients was 2.9±1.0 years.

Baseline Characteristics

The baseline characteristics of the two groups are presented in Table 1. The majority of the patients were male, Caucasian, with severe LV dysfunction (mean LVEF=27%) of ischemic etiology and with NYHA class II symptoms. Medications were well balanced between the two groups. Patients on enalapril had a higher prevalence of previous myocardial infarction, and there was a trend toward an increase in current smoker status (p=0.072).

Development of Atrial Fibrillation

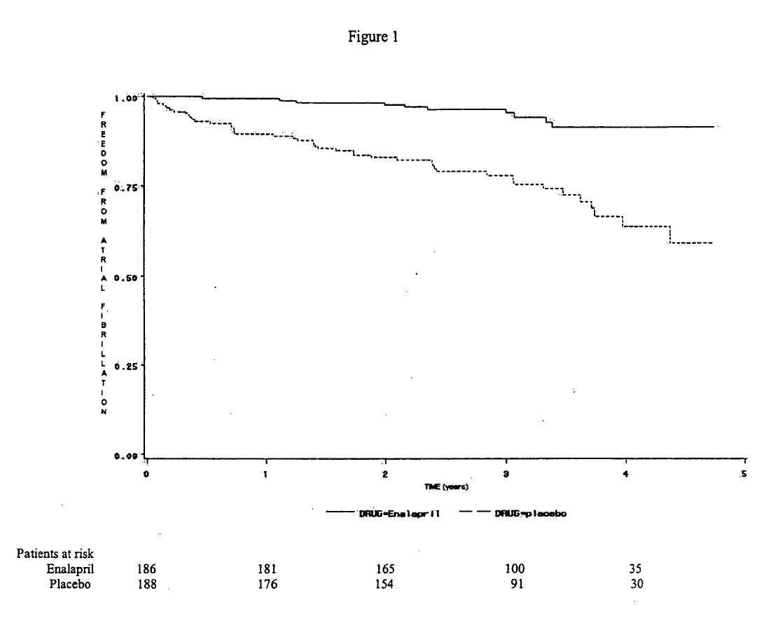

A total of 1491 ECGs were examined, 693 in the placebo group and 798 in the enalapril group (3.7±4.1 and 4.3±5.0 ECGs/patient respectively, p=NS). Similarly, 43 Holters were performed: 19 and 24 in the placebo and enalapril groups, p=NS. A total of 55 patients presented≧1 episode of AF during the 2.9 years of follow-up, 10 (5.4%) in the enalapril group and 45 (24%) in the placebo group (p<0.001), an absolute risk reduction of 18.6%. A brief description of the episodes is provided on Table 2. The majority were paroxysmal and required hospitalization for worsening HF. Despite the new onset of AF in these patients, electrical cardioversion was only performed in a minority. During follow-up, the probability of remaining in sinus rhythm was significantly higher with enalapril than with placebo (p<0.0001 FIG. 1). By Cox multivariate analysis (Table 3), allocation to enalapril was the most powerful predictor for reduction in the incidence of AF (Hazard ratio (HR)=0.22; 95% CI:0.11-0.44; p<0.0001). Although the numbers are small, the presence of an ischemic etiology for LV dysfunction also had an impact on the risk of developing AF (HR=4.9; 95% CI: 2.32-10.41; p<0.0001). Age, history of supraventricular arrhythmia and diuretics use tended to increase the risk of developing AF without reaching significance in the multivariate analysis.

Since the baseline characteristics suggested a trend toward a higher prevalence of supraventricular arrhythmia before randomization in the placebo group (7.5% versus 3.8%, p=0.121), the analysis of the effect of enalapril on AF incidence after excluding patients (n=21) with a history of supraventricular arrhythmia at baseline was repeated.

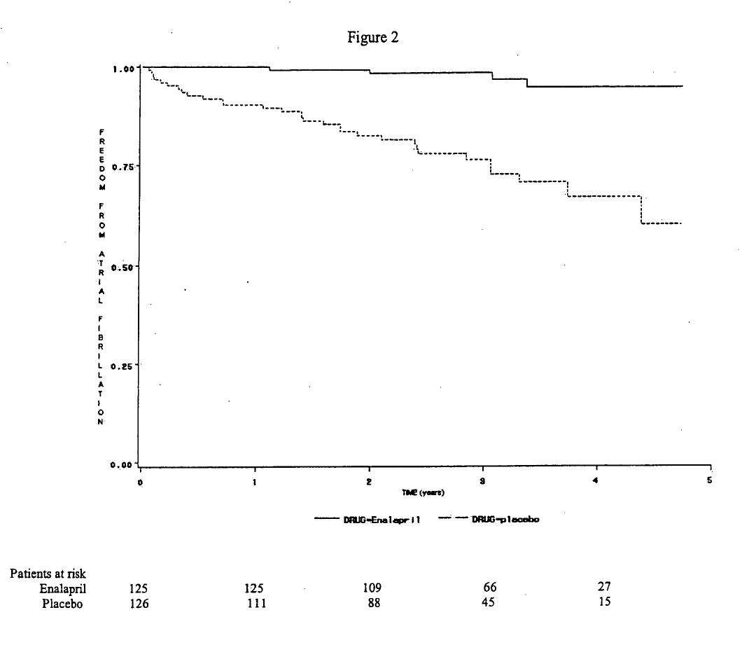

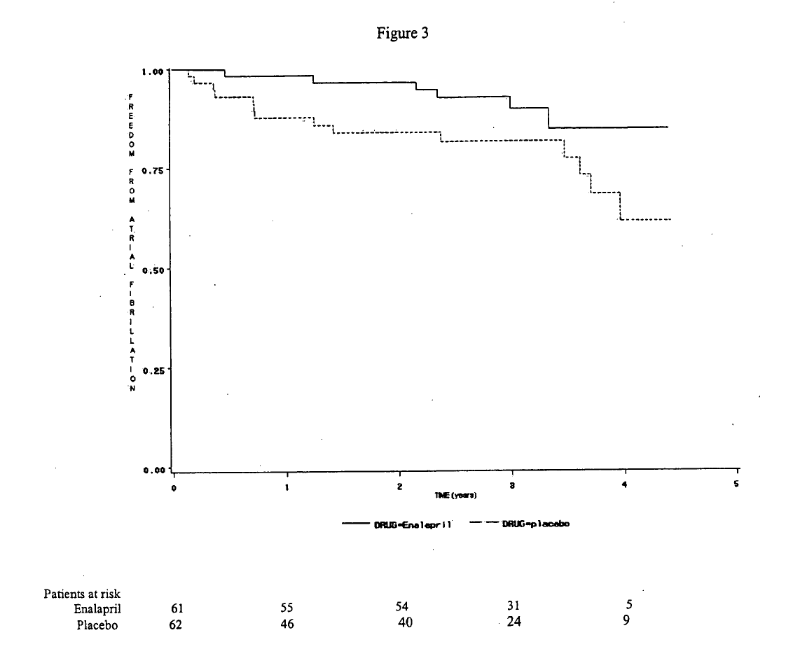

Results remained unchanged, with significantly less patients developing AF with enalapril (8 patients, 4.5%) than placebo (40 patients (23%); p<0.0001). The analysis was further stratified according to baseline functional status by analyzing the effect of enalapril on the incidence of AF in the two trial arms (prevention and treatment) separately. The beneficial effect of enalapril on the development of AF seemed more marked in the less symptomatic patients: in the SOLVD prevention arm, 4 patients (3.2%) developed AF in the enalapril group versus 31 patients (24.6%) in the placebo group (p<0.0001) whereas in the treatment arm, 6 patients (9.8%) developed AF with enalapril versus 14 (22.6%) with placebo (P=0.055). Kaplan-Meier curves for time to occurrence of AF in the two trial arms are shown in FIGS. 2 and 3.

Discussion

The above results demonstrate that chronic ACEi therapy with enalapril markedly reduces the risk of developing AF in patients with LV dysfunction. The findings extend the numerous beneficial effects of ACEi in patients with HF to the prevention of AF. This study is believed to be the first to demonstrate a reduction in the incidence of AF with ACEi in a chronic heart failure (CHF) population. Pedersen and coworkers have shown a reduction in the occurrence of AF with trandolapril (versus placebo) shortly after an acute myocardial infarction (3-7 days).¹⁶ Although LV function was depressed in their patients (mean LVEF=33%), treatment was started at the time when structural myocardial changes were occurring, and this may not reflect the CHF situation in which elevated left atrial pressure has been present for a prolonged period of time; this can at least partly explain the small absolute risk reduction (2.5%) on the incidence of AF observed during the 2-4 years of follow-up in TRACE (TRAndolapril Cardiac Evaluation). Furthermore, it is not clear whether these findings reflect a direct effect on atrial structural remodeling or are the result of the attenuation by ACEi of the left ventricular remodeling that occurs after an acute myocardial infarction.¹⁷

The mechanisms by which ACE inhibition exerts its protective effect against AF development in HF are not completely understood. A possible explanation may reside in the inhibition of the neurohormonal activation that occurs in CHF and parallels the severity of the disease. The renin-angiotensin-aldosterone system is involved in many events which could promote AF. Angiotensin-II is a potent promoter of fibrosis, leading to cardiac fibroblast proliferation and reduced collagenase activity.¹⁸⁻²⁰ Among the underlying effectors through which angiotensin-II exerts its action, Mitogen-Activated Protein Kinases (MAPKs), and specifically Extracellular signal-Regulated Kinase (ERK) seem to play a major role. Increased atrial expression of ACE and ERK have been demonstrated in experimental HF¹⁰ and in the atrial tissue of patients with a history of AF,²¹ together with AT₁ receptor downregulation and AT₂ upregulation.²² When these patients were treated with ACEi, the amount of activated ERK2 was reduced, which suggests a causal relationship. Experimentally, the atrial structural changes in HF induced by rapid ventricular pacing are attenuated when the ACE enalapril is given at the onset of pacing and the animals followed for 5 weeks.¹¹ This is accompanied by a significant reduction in atrial fibrosis and decreased vulnerability of these animals to AF. Whether this represents a direct effect of ACEi on the atrial fibrotic process or is just a consequence of decreased left atrial pressure induced by enalapril is unclear. Angiotensin-II causes an increase in atrial pressure,²³ and increased levels of atrial AT₁ receptors mRNA have also been demonstrated in response to elevated atrial pressure.²⁴ Atrial stretch induced by increased atrial pressure may be involved in the initiation and pathogenesis of AF through shortening of refractory period and lengthening of intra-atrial conduction time.^{25,26} Because ACEi cause a decline in both left atrial²⁷ and LV end-diastolic pressures in patients with HF,¹⁰ it is possible that these agents may decrease the vulnerability to AF simply by lowering atrial pressure and wall stress and consequently by attenuating left atrial enlargement. However, this hypothesis however seems less probable since Li and colleagues¹⁰ have shown experimentally a reduction in atrial fibrosis only with enalapril despite a similar decrease in left atrial pressure with hydralazine/isosorbide.

In the failing human heart, neurohormonal activation, LV remodeling, elevated left atrial pressure, and atrial fibrosis probably interact to provide a pathophysiological substrate for AF, which can thus be, at least partially, reversible with ACEi therapy.

Among other potentially beneficial mechanisms, a direct antiarrhythmic effect of ACEi on AF development cannot be excluded. Angiotensin-II seems to contribute directly to atrial electrical remodeling even in the absence of HF. The shortening of the atrial refractory period that occurs during rapid atrial pacing becomes more marked in the presence of angiotensin-II but was prevented by treatment with candesartan or captopril.²³ In patients with persistent AF, a beneficial effect of irbesartan on AF recurrence was observed when it was started 3 weeks before electrical cardioversion and combined with amiodarone.²⁸ Most of the benefit of the AT₁ receptor blocker occurred during the first 2 months after conversion, suggesting a role for irbesartan on the atrial electrical remodeling process occurring after cardioversion. The rapidly diverging Kaplan-Meier curves in the present study also suggest that enalapril acted in part through functional changes. Finally, enalapril seemed more effective in preventing AF in the least symptomatic population. Whether these differences reflect atrial structural changes that are potentially reversible in the least symptomatic patients or are simply caused by chance (because of the small number of patients involved), remains unknown. Taken together, these experimental and clinical studies suggest that treatment interfering with the renin-angiotensin system (with either ACEi or angiotensin 11 receptors blockers) have protective effects against AF development, acting through various possible mechanisms in HF patients.

Clinical Implications

Heart failure promotes AF and the latter increases the risk of thromboembolism²⁹, compromises cardiac function and increases mortality in patients with concomitant HF. Preventing AF with ACEi may thus improve the short and long term prognosis of patients with CHF, by breaking this vicious cycle and avoiding the potential risk of anti arrhythmic agents. It may also be speculated that the stroke prevention effect of ramipril obtained in the HOPE (Heart Outcomes Prevention Evaluation) study may be due at least partly to reduction in the incidence of AF in their high risk population.³⁰ With an absolute risk reduction of 18.6% when enalapril is given to HF patients, 5 patients with CHF would need to be treated for 2.9 years to prevent one episode of AF.

It should be noted that this study is a retrospective analysis of SOLVD, and the ECGs and Holter monitoring were not collected as an integral part of the studies.

Nevertheless, all the available data, regardless of the settings in which they were obtained (during hospitalizations, clinical, research or emergency room visits), were analyzed prospectively and interpreted carefully by a single experienced cardiologist, blinded to treatment allocation.

Although the present invention has been described hereinabove by way of preferred embodiments thereof, it can be modified without departing from the spirit, scope and nature of the subject invention, as defined in the appended claims.

TABLE 1
Baseline Characteristics of the Patients in the Two Groups

| | Placebo | Enalapril | |
|---|---|---|---|
| Characteristics | (n = 188) | (n = 186) | P |
| Age (years) | 57.4 ± 9.7 | 56.7 ± 9.7 | 0.499 |
| Male(%) | 91.0 | 89.8 | 0.700 |
| Weight(kg) | 76.4 ± 12.6 | 76.5 ± 11.5 | 0.947 |
| Ethnic origin (%) | | | |
| Caucasian | 98.4 | 100.0 | |
| Others | 1.6 | 0.0 | |
| Systolic blood pressure (mmHg) | 128.3 ± 17.1 | 128.0 ± 17.4 | 0.860 |
| Diastolic blood pressure (mmHg) | 79.4 ± 8.3 | 78.4 ± 8.7 | 0.267 |
| Pulse pressure (mmHg) | 48.9 ± 14.0 | 49.6 ± 13.4 | 0.640 |
| Heart rate (BPM) | 74.5 ± 10.4 | 74.6 ± 9.8 | 0.920 |
| NYHA class (%) | | | |
| 1 | 27.1 | 28.5 | |
| 2 | 63.3 | 65.1 | 0.536 |
| 3 | 9.6 | 6.5 | |
| Current smoking (%) | 35.9 | 45.5 | 0.072 |
| History of (%) | | | |
| Hypertension | 18.6 | 21.5 | 0.485 |
| Diabetes mellitus | 22.3 | 18.8 | 0.399 |
| SV arrhythmia | 7.5 | 3.8 | 0.121 |
| VT | 6.9 | 10.2 | 0.253 |
| Previous MI | 86.7 | 93.6 | 0.026 |
| Primary cause | | | |
| of LV dysfunction (%) | | | |
| Ischemic | 93.1 | 94.6 | 0.527 |
| Other | 4.3 | 4.3 | |
| Unknown | 2.7 | 1.1 | |
| Ejection fraction (%) | 26.7 ± 6.3 | 26.6 ± 6.7 | 0.869 |
| Serum potassium (meq/l) | 4.3 ± 0.4 | 4.3 ± 0.4 | 0.539 |
| Serum creatinine (mg/dl) | 1.1 ± 0.2 | 1.1 ± 0.2 | 0.858 |
| Drug therapy (%) | | | |
| Antiarrhythmic | 4.8 | 4.8 | 0.981 |
| Beta-blockers | 21.3 | 20.4 | 0.840 |
| Diuretics | 46.8 | 44.1 | 0.597 |
| Digitalis | 37.2 | 30.7 | 0.178 |
| Calcium-channel-blockers | 44.4 | 40.7 | 0.552 |
| Antiplatelet | 37.2 | 41.9 | 0.352 |

Values are presented as mean ± SD, or percentage.
NYHA = New York Heart Association;
LV = left ventricular;
SV = supraventricular;
VT = ventricular tachycardia; and
MI = myocardial infarction.

TABLE 2
Atrial Fibrillation Characteristics in the Two Groups

| Characteristics of Atrial | Placebo | Enalapril |
|---|---|---|
| Fibrillation | (n = 45) | (n = 10) |
| Detection: 12-leads ECG | 44 (97.8%) | 10 (100%) |
| Detection: Holter monitoring | 1 (2.2%) | 0 (0%) |
| Paroxysmal | 43 (95.6%) | 8 (80%) |
| Persistent | 2 (4.4%) | 2 (20%) |
| Requiring: hospitalization | 23 (51.1%) | 8 (80%) |
| Requiring: electrical cardioversion | 5 (11.1%) | 1 (10%) |

TABLE 3
Univariate and Multivariate Analysis of Variables
Influencing AF Development
Univariate Analysis

| Variables | P |
|---|---|
| At baseline | |
| Age | 0.042 |
| Sex | 0.853 |
| NYHA class | 0.174 |
| History: SV arrhythmia | 0.044 |
| History: Ventricular tachycardia | 0.360 |
| Ischemic etiology of LV dysfunction | <0.0001 |
| Diabetes | 0.499 |
| EF | 0.995 |
| Δ EF | 0.432 |
| Time-dependent | |
| SBP | 0.212 |
| Δ SBP | 0.838 |
| DBP | 0.780 |
| Δ DBP | 0.580 |
| Pulse Pressure | 0.189 |
| Serum potassium | 0.054 |
| Drug therapy | |
| Digitalis | 0.158 |
| Diuretics | 0.015 |
| Potassium sparing diuretic | 0.106 |
| Antiarrhythmic | 0.417 |
| Beta-blockers | 0.359 |
| Calcium-channel blockers | 0.116 |
| Antiplatelet | 0.237 |

Multivariate Analysis

| Variables | P value | Hazard ratio |
|---|---|---|
| Drug therapy: enalapril | <0.0001 | 0.220 |
| Ischemic etiology of LV dysfunction | <0.0001 | 4.902 |
| Age | 0.065 | 1.029 |
| History: SV arrhythmia | 0.067 | 2.245 |
| Diuretics | 0.072 | 1.749 |

NYHA indicates New York Heart Association;
SV, supraventricular;
EF, ejection fraction;
Δ EF, EF at baseline − EF at the end of the study;
SBP, systolic blood pressure;
Δ SBP, SBP at baseline − SBP at the end of the study;
DBP, diastolic blood pressure; and
Δ DBP, DBP at baseline − DBP at the end of the study.

### LIST OF REFERENCES

1. http://www.aboutatrialfibrillation.com

2. Carson P E, Johnson G R, Dunkman W B, et al. The influence of atrial fibrillation on prognosis in mild to moderate heart failure: The V-HeFT Studies. Circulation. 1993;87(Suppl.VI):VI-102-VI-110.

3. Middlekauff H R, Stevenson W G, Stevenson L W. Prognostic significance of atrial fibrillation in advanced heart failure: a study of 390 patients. Circulation. 1991;84:4048.

4. Pozzoli M, Cioffi G, Traversi E, et al. Predictors of primary atrial fibrillation and concomittant clinical and hemodynamic changes in patients with chronic heart failure: a prospective study in 344 patients with baseline sinus rhythm. J Am Coll Cardiol. 1998;32:197-204.

5. Dries D L, Exner D V D, Gersh B J, et al. Atrial fibrillation is associated with an increased risk for mortality and heart failure progression in patients with asymptomatic and symptomatic left ventricular systolic dysfunction: a retrospective analysis of the SOLVD trials. J Am Coll Cardiol. 1998;32:695-703.

6. Bourassa M G, Gurné O, Bangdiwala S I, et al. Natural history and patterns of current practice in heart failure. J Am Coll Cardiol. 1993;22(Suppl.A):14A-19A.

7. Mathew J, Hunsberger S, Fleg J, et al. Incidence, predictive factors, and prognostic significance of supraventricular tachyarrhythmias in congestive heart failure. Chest. 2000; 118:914-922.

8. Li D, Fareh S, Leung T K, et al. Promotion of atrial fibrillation by heart failure in dogs: atrial remodeling of a different sort. Circulation. 1999;100:87-95.

9. Shi Y, Ducharme A, Li D, et al. Remodeling of atrial dimensions and emptying function in canine models of atrial fibrillation. Cardiovasc Res. 2001;52:217-225.

10. Li D, Shinagawa K, Pang L, et al. Effects of angiotensin-converting enzyme inhibition on the development of the atrial fibrillation substrate in dogs with ventricular tachypacing-induced congestive heart failure. Circulation. 2001; 104:2608-2614.

11. Shi Y, Li D, Tardif J C, et al. Enalapril effects on atrial remodeling and atrial fibrillation in experimental congestive heart failure. Cardiovasc Res. 2002;54:456-461.

12. The SOLVD Investigators. Effect of enalapril on survival in patients with reduced left ventricular ejection fractions and congestive heart failure. N Engl J. Med. 1991;325:293-302.

13. The SOLVD Investigators. Effect of enalapril on mortality and the development of heart failure in asymptomatic patients with reduced left ventricular ejection fractions. N Engl J. Med. 1992;327:685-691.

14. The SOLVD Investigators. Studies of Left Ventricular Dysfunction (SOLVD)-Rationale, design and methods: two trials that evaluate the effect of enalapril in patients with reduced ejection fraction. Am J Cardiol. 1990;66:315-322.

15. Fuster V, Ryden L E, Asinger R W et al. ACC/AHA/ESC guidelines for the management of patients with atrial fibrillation: executive summary. J Am Coll Cardiol. 2001;38:1231-1266.

16. Pedersen O D, Bagger H, Køber L, et al. Trandolapril reduces the incidence of atrial fibrillation after acute myocardial infarction in patients with left ventricular dysfunction. Circulation. 1999; 100:376-80.

17. St John Sutton M, Pfeffer M A, Plappert T et al. Quantitative two-dimensional echocardiographic measurements are major predictors of adverse cardiovascular events after acute myocardial infarction. The protective effects of captopril. Circulation. 1994;89:68-75.

18. Zou Y, Komuro I, Yamazaki T, et al. Cell type-specificangiotensin 1′-evoked signal transduction pathways: critical roles of G_{βγ} subunit, Src family, and Ras in cardiac fibroblasts. Circ Res. 1998;82:337-345.

19. Pagés G, Lenormand P, L'Allemain G, et al. Mitogen-activated protein kinases p42^{mapk} and 44^{mapk} are required for fibroblast proliferation. Proc Natl Acad. Sci. 1993;90:8319-8323.

20. BrilIa C G, Zhou G, Matsubara L, et al. Collagen metabolism in cultured adult rat cardiac fibroblasts: response to angiotensin 11 and aldosterone. J Mol Cell Cardiol 1994;26:809-820.

21. Goette A, Staack T, Rocken C et al. Increased expression of extracellular signal regulated kinase and angiotensin-converting enzyme in human atria during atrial fibrillation. J Am Coll Cardiol. 2000;35:1669-1677.

22. Goette A, Arndt M, Rocken C et al. Regulation of angiotensin 11 receptor subtypes during atrial fibrillation in humans. Circulation. 2000;101:2678-2681.

23. Nakashima H, Kumagai K, Urata H, et al. Angiotensin II antagonist prevents electrical remodeling in atrial fibrillation. Circulation. 2000;101:2612-2617.

24. Kaprielian R, Dupont E, Hafizi Set al. Angiotensin II receptor type 1 mRNA is upregulated in atria of patients with end-stage heart failure. J Mol Cell Cardiol. 1997;29:2299-2304.

25. Solti F, Vecsey T, Kékesi V, et al. The effect of atrial dilatation on the genesis of atrial arrhythmias. Cardiovasc Res. 1989;23:882-886.

26. Ravelli F, Allessie M. Effects of atrial dilatation on refractory period and vulnerability to atrial fibrillation in the isolated Langendorff-perfused rabbit heart. Circulation. 1997;96:1686-1695.

27. Webster M W, Fitzpatrick M A, Nicholls M G, et al. Effect of enalapril on ventricular arrhythmias in congestive heart failure. Am J Cardiol. 1985;56:566-569.

28. Madrid A H, Bueno M G, Rebollo M G et al. Use of irbesartan to maintain sinus rhythm in patients with long-lasting persistent atrial fibrillation. Circulation 2002;106:331-336.

29. Lamassa M, Di Carlo A, Pracucci G, et al. Characteristics, outcome, and care of stroke associated with atrial fibrillation in Europe. Stroke. 2001;32:392-398.

30. Bosch J, Yusuf S, Pogue J et al. Use of ramipril in preventing stroke: double blind randomised trial. BMJ. 2002;324:699-702.

What is claimed is:

1. A method for preventing the incidence of atrial fibrillation (AF) in a subject with chronic heart failure comprising the administration of a therapeutically effective amount of an ACE inhibitor.

2. A method as defined in claim 1, wherein said chronic heart failure is a result of symptomatic or asymptomatic left ventricular systolic dysfunction.

3. A method as defined in claim 2, wherein said chronic heart failure is a result of symptomatic left ventricular systolic dysfunction.

4. A method as defined in claim 1, wherein said ACE inhibitor is selected from the group consisting of: enalapril (Vasotec®), captopril (Capoten®), lisinopril (Prinivil®, Zestril®), quinapril (Accupril®), ramipril (Altace®), trandolapril (Mavick®), perindopril (Coversyl®)), and fosinopril (Monopril®).

5. A method as defined in claim 4, wherein said ACE inhibitor enalapril is administered in an amount of about 5-20 mg/day, said ACE inhibitor captopril is administered in an amount of about 150 mg/day, said ACE inhibitor lisinopril is administered in an amount of about 20 mg/day, said ACE inhibitor quinapril is administered in an amount of about 40 mg/day, said ACE inhibitor ramipril is administered in an amount of about 10 mg/day, said ACE inhibitor trandolapril is administered in an amount of about 4 mg/day, said ACE inhibitor perindopril is administered in an amount of about 8 mg/day, and said ACE inhibitor fosinopril is administered in an amount of about 20 mg/day.

6. A method as defined in claim 5, wherein said ACE inhibitor is enalapril.

7. A method for preventing the incidence of atrial fibrillation in a subject with chronic heart failure comprising the administration of a therapeutically effective amount of angiotensin 11 receptor antagonist.

8. A method as defined in claim 7, wherein said chronic heart failure is a result of symptomatic or asymptomatic left ventricular systolic dysfunction.

9. A method as defined in claim 8, wherein said chronic heart failure is a result of symptomatic left ventricular systolic dysfunction.

10. A method as defined in claim 9, wherein said angiotensin 11 receptor antagonist is selected from the group consisting of: losartan (Cozaar®), candesartan (Atacand®), irbesartan (Avapro®), telmisartan (Micardis®), valsartan (Diovan®) and eprosartan (Teveten®).

11. A method as defined in claim 10, wherein said angiotensin 11 receptor antagonist is administered in an amount of about 5-20 mg/day.

12. A method as defined in claim 11, wherein said angiotensin 11 receptor antagonist is losartan, valsartan or candesartan.
